(19) United States
(12) Patent Application Publication
(10) Pub. No.: US 2023/0240736 A1
(43) Pub. Date: Aug. 3, 2023

(54) ASSEMBLABLE CRYONEEDLE AND METHOD FOR USING SAME

(71) Applicant: LIFE BY ICE LTD., Haifa (IL)

(72) Inventor: Yaron HAR-SHAI, Haifa (IL)

(21) Appl. No.: 17/592,475

(22) Filed: Feb. 3, 2022

(51) Int. Cl. A61B 18/02

(52) U.S. Cl. CPC A61B 18/02; A61B 2018/0293; A61B 2018/00577

(57) ABSTRACT

There is provided herein an assemblable cryoneedle for performing a cryo-treatment, particularly but not limited to, for cryo-treatment of keloids and scars. According to some embodiments, the assemblable cryoneedle includes an outer elongated needle having a sealed distal portion, an inner elongated needle, and a connector, configured to connect and allow fluid communication therebetween. The inner elongated needle is configured to be inserted into the outer elongated needle, forming a double lumen configuration. The outer elongated needle is configured to be inserted into a treatment area of a subject.

### TECHNICAL FIELD

The present disclosure relates generally to an assemblable cryoneedle and a method for performing a cryosurgery.

### BACKGROUND

Cryosurgery, also known as cryotherapy and cryoablation, is a medical procedure for treating an abnormal tissue by cooling/freezing. It has been widely studied for treating cancer tumors, including prostate cancer, kidney cancer, and other tumors. Currently, cryosurgery is widely applied in dermatology for treating skin conditions, such as scars and keloids.

Keloids are proliferative scars which occur due to an abnormal scar and/or wound healing, forming firm, bulky and excessive scar tissue extending beyond the original scar and/or wound borders. Thus, causing physical symptoms such as inconvenience, itchiness, tenderness, irritation, and pain, and negatively impacting the emotional well-being and quality of life. Due to the prolonged expending periods (months and even years), as well as the extremely high tendency of recurrence, keloid treatment methods such as steroid injection, surgical excision, radiation or laser therapy, or any combination thereof, demonstrate low treatment efficacy. Currently, cryosurgery is considered probably the most effective medical procedure for treating skin lesions such as keloids.

During cryosurgery, a cryoneedle is inserted into a treatment area, exposing cells in the treatment area to a low temperature, thereby extracting heat (thermal energy) from the cells in the treatment area to the cryoneedle, and decreasing the temperature of the cells until reaching thermal equilibrium (i.e. equalizing the temperature of the cryoneedle and the cells) thus forming ice crystals inside the cells which, in turn, is followed by mechanical disruption and death of the cells and obliteration of blood vessels. Most of such procedures typically last about up to 10 minutes, and hence currently applied cryoneedles are usually designed for relatively short cryoablative treatments. Nonetheless, the optimal cryoablative “dose” (in terms of treatment area temperature and cooling/freezing duration) for treating tissue is yet to be established, due to the differences in the anatomy of the treatment area, blood vessels (serving as a heat source), and medical practices.

Accordingly, there is a need in the art for cryoneedles capable of withstanding low temperatures for prolonged periods of time.

### SUMMARY

Aspects of the disclosure, according to some embodiments thereof, relate to cryoneedles and methods for performing a cryo-treatment.

According to some embodiments, there is provided herein an assemblable cryoneedle for performing a cryo-treatment, particularly but not limited to, for cryo-treatment of keloids and scars. According to some embodiments, the assemblable cryoneedle includes an outer elongated needle having a sealed distal portion, an inner elongated needle, and a connector, configured to connect and allow fluid communication therebetween. The inner elongated needle is configured to be inserted into the outer elongated needle, forming a double lumen configuration. The outer elongated needle is configured to be introduced into a treatment area of a subject.

According to some embodiments, there is further provided herein a kit for performing cryo-treatments. According to some embodiments, the kit includes one or more assemblable cryoneedles. According to some embodiments, the kit includes a splitter for facilitating simultaneous connecting of a plurality of cryoneedles to a single cryogen source. According to some embodiments, the kit includes a non-elastic fastener with a quick release mechanism configured to maintain continuous supply of the cryogen without a need of a user holding a trigger of the cryogen source in an open position. According to some embodiments, the kit includes a trocar, preferably with a sharp sealed distal tip, configured to be introduced into the treatment area prior to inserting the cryoneedle, thereby facilitating the cryoneedle insertion.

According to some embodiments, there is provided herein a kit for cryo-treatment of a subject in a need thereof, the kit includes: one or more assemblable cryoneedles as disclosed herein in accordance with some embodiments; and at least one of: a non-elastic fastener configured to maintain continuous supply of the cryogen without a need of a user holding a trigger of the cryogen source in an open position; a splitter for facilitating simultaneous connecting of a plurality of cryoneedles to a single cryogen source; and a trocar, preferably with a sharp sealed distal tip, configured for introducing into the treatment area prior to inserting the cryoneedle therein, to facilitate the cryoneedle insertion.

While most of the prior art cryoneedles are welded cryoneedles which are commonly designed for cryo-treatments durations of several seconds or a few minutes, the herein disclosed assemblable cryoneedle and kit advantageously allow performing long cryo-treatments (e.g., for several hours) to the subject without the need of replacing the cryoneedle, with or without replacing the cryogen source. This is achieved by the unique structure of the assemblable cryoneedle and its manufacturing method, which, advantageously, is devoid of welding and metal turning, thereby minimizing cryogen leaking from the assemblable cryoneedle, reducing production costs, and increasing the safety of a subject and a user.

According to some embodiments, the disclosed herein assemblable cryoneedle, and in particular the outer elongated needle, includes an integrally formed distal end, thus facilitating continuous, adjustable, and safe delivery of the cryogen therethrough. Advantageously, the integrally formed distal end may include a needle/cutting tip which cannot be detached from the assemblable cryoneedle, thus enhancing the safety of the procedure.

Furthermore, the herein disclosed assemblable cryoneedle, according to some embodiments, is configured to facilitate approaching the treatment area by a bent assemblable cryoneedle without reducing the heat exchange rate between the assemblable cryoneedle and the treatment area.

According to some embodiments, the design of the assemblable cryoneedle advantageously further considers the safety and comfort of the user (e.g., medical staff performing the cryosurgery) during the long treatment by coupling the assemblable cryoneedle with insulating gripping elements, to facilitate prolonged gripping and prevent frostbite injuries.

According to some embodiments, there is provided herein an assemblable cryoneedle including: an outer elongated needle, configured to be inserted into a treatment area of a subject, the outer elongated needle comprising a sealed distal portion and a proximal portion comprising an opening; an inner elongated needle, configured to be inserted into the outer elongated needle, the inner elongated needle includes: a proximal portion, having a proximal opening and configured to be connected to a cryogen source, and a distal portion, having a distal opening, wherein the proximal and distal openings of the inner elongated needle define a lumen there between; and a connector including: a distal portion having a distal opening; a proximal portion having a proximal opening, wherein the connector's proximal and distal openings define a lumen there between; and an outlet port fluidly connected to the lumen, the connector is configured to detachably couple the outer elongated needle with the inner elongated needle, wherein the connector is configured to facilitate flow of a cryogen from the proximal portion to the distal portion of the inner elongated needle, back from the sealed distal portion of the outer elongated needle, and out through the outlet port of the connector.

According to some embodiments, there is provided herein an assemblable cryoneedle including an outer elongated needle, configured to be inserted into a treatment area of a subject, the outer elongated needle includes a sealed distal portion and a proximal portion having an opening. According to some embodiments, the assemblable cryoneedle further includes an inner elongated needle, configured to be inserted into the outer elongated needle, the inner elongated needle includes a proximal portion, having a proximal opening and configured to be connected to a cryogen source, and a distal portion, having a distal opening. According to some embodiments, the proximal and distal openings of the inner elongated needle define a lumen there between. According to some embodiments, the assemblable cryoneedle includes a connector, the connector includes a distal portion having a distal opening, a proximal portion having a proximal opening, wherein the connector's proximal and distal openings define a lumen there between. According to some embodiments, the connector may include an outlet port fluidly connected to the lumen. According to some embodiments, the connector is configured to detachably couple the outer elongated needle with the inner elongated needle. According to some embodiments, the connector is configured to facilitate flow of a cryogen from the proximal portion to the distal portion of the inner elongated needle, back from the sealed distal portion of the outer elongated needle, and out through the outlet port of the connector.

According to some embodiments, there is provided herein an assemblable cryoneedle including: an outer elongated needle, configured to be inserted into a treatment area of a subject, the outer elongated needle comprising a sealed distal portion and a proximal portion comprising an opening; an inner elongated needle, configured to be inserted into the outer elongated needle, the inner elongated needle includes: a proximal portion, having a proximal opening, and a distal portion, having a distal opening, wherein the proximal and distal openings of the inner elongated needle define a lumen there between; and a connector including: a distal portion having a distal opening; a proximal portion having a proximal opening, wherein the connector's proximal and distal openings define a lumen there between; and an inlet port fluidly connected to the lumen, the connector is configured to detachably couple the outer elongated needle with the inner elongated needle, wherein the connector is configured to facilitate flow of a cryogen from the proximal portion to the distal portion of the outer elongated needle, back from the sealed distal portion of the outer elongated needle into the distal portion of the inner elongated needle, and out through the proximal portion of the inner elongated tube.

According to some embodiments, there is provided herein an assemblable cryoneedle including an outer elongated needle, configured to be inserted into a treatment area of a subject, the outer elongated needle includes a sealed distal portion and a proximal portion having an opening. According to some embodiments, the assemblable cryoneedle further includes an inner elongated needle, configured to be inserted into the outer elongated needle, the inner elongated needle includes a proximal portion, having a proximal opening and configured to be connected to a cryogen source, and a distal portion, having a distal opening. According to some embodiments, the proximal and distal openings of the inner elongated needle define a lumen there between. According to some embodiments, the assemblable cryoneedle includes a connector, the connector includes a distal portion having a distal opening, a proximal portion having a proximal opening, wherein the connector's proximal and distal openings define a lumen there between. According to some embodiments, the connector may include an inlet port fluidly connected to the lumen. According to some embodiments, the connector is configured to detachably couple the outer elongated needle with the inner elongated needle. According to some embodiments, the connector is configured to facilitate flow of a cryogen from the proximal portion to the distal portion of the outer elongated needle, back from the sealed distal portion of the outer elongated needle into the distal portion of the inner elongated needle, and out through the proximal portion of the inner elongated tube.

According to some embodiments, the proximal portion of the outer elongated needle is connectable to the distal portion of the connector, and the proximal portion of the inner elongated needle is connectable to the proximal portion of the connector.

According to some embodiments, the outer elongated needle, the inner elongated needle and/or the connector are manufactured by CNC machining.

According to some embodiments, the sealed distal portion of the outer elongated needle further includes a sharp tip configured to penetrate the treatment area. According to some embodiments, the sharp tip may include a cutting tip, e.g., conventional, tapered, side cutting, K needle or any combination thereof

According to some embodiments, the sharp tip is integrally formed with the outer elongated needle.

According to some embodiments, at least a portion of the outer elongated needle and the inner elongated needle are bent, maintaining a fluid flow circulation capacity between an outer surface of the inner elongated needle and an inner surface of the outer elongated needle and inside the inner elongated needle.

According to some embodiments, the proximal portion of the inner elongated needle and/or the inlet/outlet port of the connector is configured to be connected directly or indirectly to a cryogen source via a splitter, the splitter facilitates concomitantly connecting a plurality of cryoneedles to a single cryogen source and preserving the working pressure to each cryoneedle. According to some embodiments, the working pressure of each of the cryoneedles is in the range of about 10-12 psi.

According to some embodiments, the proximal portion of the outer elongated needle is detachably connectable to the distal portion of the connector by screwing. According to some embodiments, a screwing segment is couplable to the proximal portion of the outer elongated needle by mechanical pressure, adjusted by tolerance values there between.

According to some embodiments, the proximal portion of the inner elongated needle is detachably connectable to the proximal portion of the connector by screwing. According to some embodiments, a screwing segment is couplable to the proximal portion of the inner elongated needle by mechanical pressure, adjusted by tolerance values there between.

According to some embodiments, the connector further includes one or more flat outer surfaces, to facilitate coupling and retaining of the connector to the proximal portion of the outer elongated needle.

According to some embodiments, at least a portion of the proximal portion of the outer elongated needle is circumferentially expanded (comprising a one or more flat outer surfaces) to facilitate coupling and retaining the proximal portion of the outer elongated needle to the connector.

According to some embodiments, the connector further includes an interlocking component on an outer surface thereof, the interlocking component is configured to secure the proximal portions of the inner elongated needle and/or the outer elongated needle to the connector.

According to some embodiments, the proximal portion of the inner elongated needle further includes one or more circumferentially expanded cone-shaped collars, to facilitate direct or indirect connection and sealing of the inner elongated needle to the cryogen source.

According to some embodiments, the outlet/inlet port is integrally formed with the connector. According to some embodiments, the outlet/inlet port is couplable to the connector by mechanical pressure, adjusted by tolerance values there between.

According to some embodiments, the outlet port of the connector is configured to receive an extension tube to distance the exiting cryogen from the subject and/or a user.

According to some embodiments, the proximal portion of the inner elongated needle is configured to receive an extension tube to distance the exiting cryogen from the subject and/or a user.

According to some embodiments, the connector further includes one or more thermally insulating gripping elements configured to facilitate gripping of the cryoneedle by a user. According to some embodiments, the one or more thermally insulating gripping elements include two gripping elements configured to cover the connector and facilitate penetrating the cryoneedle into the treatment area, gripping of the cryoneedle by the user, and withdrawing the cryoneedle from the treatment area. According to some embodiments, the one or more thermally insulating gripping elements include two gripping elements configured to snap fit and cover the connector.

According to some embodiments, there is provided herein a method for performing a cryosurgery, the method including: providing an assemblable cryoneedle as disclosed herein in accordance with some embodiments; contacting the assemblable cryoneedle with the treatment area of the subject or inserting the assemblable cryoneedle to the treatment area of the subject; supplying a cryogen from a cryogenic source through the assemblable cryoneedle, such that the cryogen flows from the proximal portion to the distal portion of the inner elongated needle, back from the sealed distal portion of the outer elongated needle, and out through the outlet port of the connector; and retracting the assemblable cryoneedle.

According to some embodiments, there is provided herein a method for performing a cryosurgery, the method including: providing an assemblable cryoneedle as disclosed herein in accordance with some embodiments; contacting the assemblable cryoneedle with the treatment area of the subject or inserting the cryoneedle to the treatment area of the subject; supplying a cryogen from a cryogenic source through the cryoneedle, such that the cryogen flows from the inlet port of the connector through the proximal portion of the outer elongated needle towards the distal portion of the outer elongated needle, back from the sealed distal portion of the outer elongated needle, and out through the inner elongated needle of the connector; and retracting the assemblable cryoneedle.

According to some embodiments, the step of inserting and/or retracting the assemblable cryoneedle is performed by rotating the cryoneedle. According to some embodiments, the assemblable cryoneedle may be repetitively rotated in circular back-and-forth motion along an angle-limited circular path, e.g., by partially rotating the assemblable cryoneedle. According to some embodiments, the step of retracting the assemblable cryoneedle is performed by rotating the cryoneedle clockwise to break the screwing connection (between the outer elongated needle and the connector) and render the cryoneedle a single use cryoneedle.

According to some embodiments, the step of retracting/withdrawing the cryoneedle further comprises breaking the connection between the outer elongated needle and the connector, thereby rendering the cryoneedle disposable.

Alternatively, or additionally, according to some embodiments, the step of inserting and/or retracting the assemblable cryoneedle may be performed by rotating the cryoneedle counterclockwise.

According to some embodiments, the cryogen source is a container comprising liquid nitrogen.

According to some embodiments, the disclosed methods further include providing a non-elastic fastener configured to maintain continuous supply of the cryogen for minutes to hours without a need of a user holding a trigger of the cryogen source in an open position. According to some embodiments, the fastener includes a quick release mode to immediately cease the cryogen supply.

According to some embodiments, the disclosed methods further include providing a trocar, preferably with a sharp sealed distal tip, to be introduced into the treatment area prior to inserting the cryoneedle into the treatment area, to facilitate the cryoneedle insertion. According to some embodiments, the outer diameter of the outer elongated needle is larger than the outer diameter of the trocar.

According to some embodiments, the cryosurgery is performed for treating hypertrophic scars and/or keloids. According to some embodiments, the cryosurgery is performed for treating scars.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In block diagrams and flowcharts, optional elements/components and optional stages may be included within dashed boxes.

In the figures:

FIG. 1A, shows a schematic illustration of a side view of an outer elongated needle, according to some embodiments;

FIG. 1B, shows a schematic illustration of a perspective side view of a proximal portion of the outer elongated needle shown in FIG. 1A, according to some embodiments;

FIG. 2A, shows a schematic illustration of a side view of an inner elongated needle, according to some embodiments;

FIG. 2B, shows a schematic illustration of a perspective side view of a proximal portion of the inner elongated needle shown in FIG. 2A, according to some embodiments;

FIG. 3A, shows a schematic illustration of a perspective side view of a connector, according to some embodiments;

FIG. 3B, shows a schematic illustration of a cross-sectional side view of the connector shown in FIG. 3A, according to some embodiments

FIG. 4A shows a schematic illustration of a perspective view an assemblable cryoneedle, according to some embodiments;

FIG. 4B shows a schematic illustration of a side view of the assemblable cryoneedle shown in FIG. 4A, according to some embodiments;

FIG. 4C shows a schematic illustration of a top view of the assemblable cryoneedle shown in FIG. 4A, according to some embodiments;

FIG. 5A shows a schematic illustration of a cross-sectional side view of an assemblable cryoneedle, according to some embodiments;

FIG. 5B shows a schematic illustration of a cross-sectional side view of an assemblable cryoneedle, according to some embodiments;

FIG. 6 shows a schematic illustration of a cross-sectional side view of an assemblable cryoneedle, according to some embodiments;

FIG. 7 shows a schematic illustration of a cross-sectional top view of s splitter, according to some embodiments;

FIG. 8 shows a schematic illustration of a side view of one or more thermally insulating gripping elements, according to some embodiments;

FIG. 9A shows a schematic illustration of a side view of a cryogen source system for supplying a cryogen fluid, according to some embodiments;

FIG. 9B shows a schematic illustration of a top view of the cryogen source system shown in FIG. 9A, according to some embodiments;

FIG. 9C shows a schematic illustration of a side view of a cryogen source system for supplying a cryogen fluid, according to some embodiments; and

FIG. 10 shows a flow chart of a method for using an assemblable cryoneedle, according to some embodiments.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the following description, various aspects of the invention will be described. For the purpose of explanation, specific details are set forth in order to provide a thorough understanding of the invention. However, it will also be apparent to one skilled in the art that the invention may be practiced without specific details being presented herein.

Furthermore, well-known features may be omitted or simplified in order not to obscure the invention.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. For example, the statement “the length of the element is equal to about 1 m” is equivalent to the statement “the length of the element is between 0.8 m and 1.2 m”. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “approximate” and “about” may be interchangeable.

In the specification and claims of the application, the terms “inserted”, “penetrated”, “introduced”, and “pierced” may be used interchangeably. According to some embodiments, introducing of the disclosed herein assemblable cryoneedle into a treatment area may include rotational motion. According to some embodiments, introducing of the disclosed herein assemblable cryoneedle into the treatment area may include repetitive circular back-and-forth motion. According to some embodiments, the assemblable cryoneedle may be repetitively rotated in circular back-and-forth motion along an angle-limited circular path.

In the specification and claims of the application, the terms “retracted”, “removed” and “withdrawn” may be used interchangeably. According to some embodiments, withdrawing of the assemblable cryoneedle from the treatment area may include repetitive circular back-and-forth motion.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods according to some embodiments may be described in a specific sequence, methods of the disclosure may include some or all of the described stages carried out in a different order. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

According to some embodiments, there is provided an assemblable cryoneedle for performing a medical procedure. According to some embodiments, the assemblable cryoneedle includes an outer elongated needle, an inner elongated needle, and a connector, configured to connect and allow fluid communication therebetween. The inner elongated needle is configured to be inserted into the outer elongated needle, forming a double lumen configuration.

According to some embodiments, the outer elongated needle and the inner elongated needle and may be concentric. According to some embodiments, the outer elongated needle may include 14 G, 15 G, 16 G, 18 G, 20 G, 22 G or any other needle size. Each possibly is a separate embodiment. According to some embodiments, the inner elongated needle may include 18 G, 20 G, 22 G or any other needle size. Each possibly is a separate embodiment.

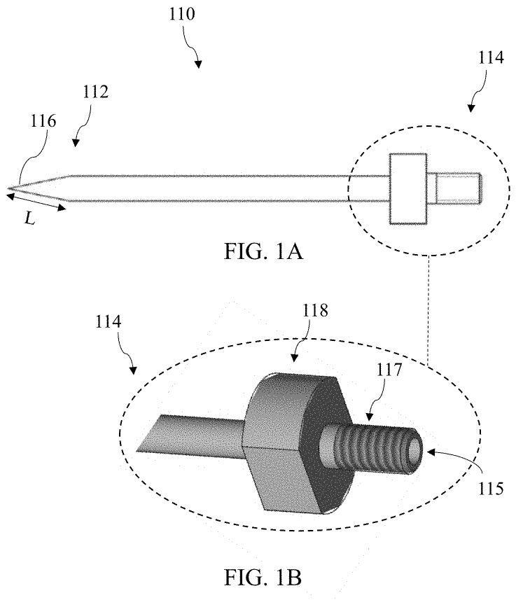

Reference is now made to FIG. 1A, which schematically illustrates a side view of an outer elongated needle 110, and FIG. 1B, which schematically illustrates a perspective side view of a proximal portion 114 thereof, according to some embodiments. According to some embodiments, outer elongated needle 110 is configured to be inserted into a treatment area of a subject. According to some embodiments, outer elongated needle 110 is configured for single use for a single subject. Put differently, outer elongated needle 110 is configured to treating a single subject in a single treatment session. According to some embodiments, outer elongated needle 110 may be used in a single treatment session for treating one treatment area (e.g., a single keloid). According to some embodiments, outer elongated needle 110 may be used in a single treatment session for treating a plurality of treatment areas (i.e., treating a plurality of keloids during the same treatment session of the same subject). According to some embodiments, outer elongated needle 110 may be disposed following the treatment session.

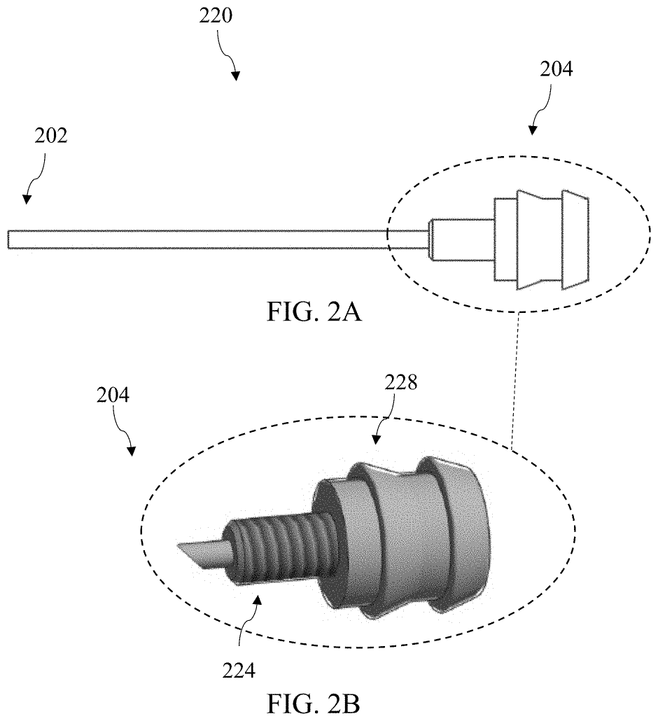

According to some embodiments, outer elongated needle 110 may include 14 G, 15 G, 16 G, 17 G, 18 G, 19 G, 20 G, 21 G, 22 G or any other needle size. Each possibly is a separate embodiment. According to some embodiments, length of outer elongated needle 110 (from a connection interface between outer elongated needle 110 and a connector (not shown) to a sealed distal portion 112) may be in the range of about 3 cm to about 15 cm. As a non-limiting example, length of outer elongated needle 110 may be about 10 cm. According to some embodiments, outer elongated needle 110 may be about 1 cm longer than an inner elongated needle (e.g., an inner elongated needle 220 depicted in FIG. 2A). According to some embodiments, outer elongated needle 110 may be about 0.5 cm longer than inner elongated needle 220. According to some embodiments, outer elongated needle 110 may be about 0.3 cm longer than inner elongated needle 220.

According to some embodiments and as depicted in FIG. 1A, outer elongated needle 110 is unbent. According to some embodiments, outer elongated needle 110 may be bent, as further elaborated. According to some embodiments, outer elongated needle 110 includes or made of thermally conductive material, such as stainless steels, metals, and the like. As a non-limiting example, outer elongated needle 110 may be made of or include 306 stainless steel. According to some embodiments, outer elongated needle 110 may be manufactured by CNC machining. Alternatively, according to some embodiments, outer elongated needle 110 may be manufactured by other suitable techniques (e.g., laser-assisted manufacturing).

According to some embodiments, outer elongated needle 110 includes a sealed distal portion 112 configured to prevent leaking of a cryogen fluid. According to some embodiments, sealed distal portion 112 includes a sharp tip 116. According to some embodiments, sharp tip 116 may include a cutting tip, e.g., conventional, tapered, side cutting, K needle or any combination thereof, to facilitate inserting and/or puncturing a treatment area of a subject.

According to some embodiments, penetrating the treatment area by outer elongated needle 110 may be facilitated by modifying a bevel angle of sealed distal portion 112, and specifically the bevel angle of sharp tip 116. According to some embodiments, the bevel angle of sharp tip 116 may be in the range of about 10-20 degrees. As a non-limiting example, the bevel angle of sharp tip 116 may be equal to about 10, 11, 12, 13, 14, 15, 16, 17, 18, 19, or 20 degrees. According to some embodiments, the bevel angle may be equal to a different value. Each possibility is a separate embodiment.

According to some embodiments, sharp tip 116 length, i.e., the length from the starting point of the bevelling until the edge of sharp tip 116 (marked by the letter L in FIG. 1A) may be in the range of about 5-10 mm. According to some embodiments, the length of sharp tip 116 may be about 5, 6, 7, 8, 9, 10 mm. According to some embodiments, the length of sharp tip 116 may be equal to a different value. Each possibility is a separate embodiment.

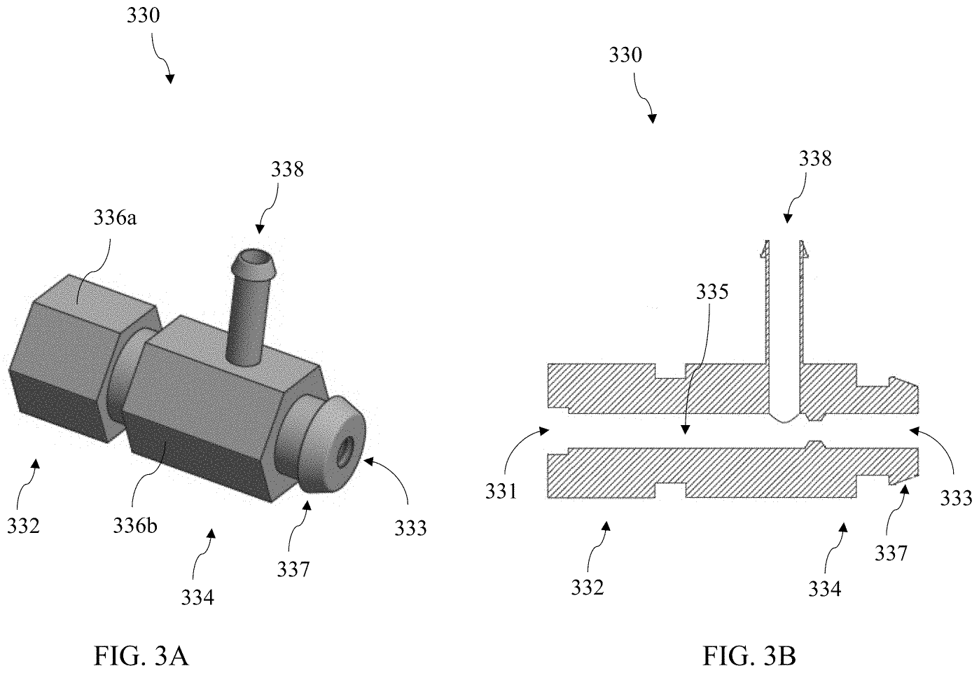

According to some embodiments, outer elongated needle 110 includes proximal portion 114 configured to be connected to a connector (shown in FIG. 3A). According to some embodiments, proximal portion 114 may be detachably connected to the connector. According to some embodiments, proximal portion 114 is connected to the connector via a screwing segment 117. According to some embodiments, screwing segment 117 may be integrally formed with outer elongated needle 110. According to some embodiments, screwing segment 117 may be couplable to the proximal portion 114 of the outer elongated needle 110 by mechanical pressure, adjusted by tolerance values there between.

According to some embodiments, proximal portion 114 includes one or more flat outer surfaces 118 to facilitate coupling and retaining the proximal portion thereof to the connector. According to some embodiments, one or more flat outer surfaces 118 may have various structures, such as flat, circular, oval, rectangular, polygon-like, or any combination thereof. Each possibility is a separate embodiment. According to some embodiments, one or more flat outer surfaces 118 may be at least partially circumferentially expended.

According to some embodiments, proximal portion 114 includes an opening 115 configured to allow cryogen fluid flow therethrough. According to some embodiments, the cryogen fluid flow may be exiting and/or entering proximal portion 114 of the outer elongated needle 110.

Reference is now made to FIG. 2A, which schematically illustrates a side view of an inner elongated needle 220, and FIG. 2B, which schematically illustrates a perspective side view of a proximal portion 204 thereof, according to some embodiments. According to some embodiments, inner elongated needle 220 is configured to be inserted into an outer elongated needle, e.g., the previously disclosed outer elongated needle 110 depicted in FIG. 1A.

According to some embodiments, inner elongated needle 220 may be configured for a single use in a single subject. According to some embodiments, inner elongated needle 220 may be used in a single treatment session for treating one treatment area (e.g., a single keloid). According to some embodiments, inner elongated needle 220 may be used in a single treatment session for treating a plurality of treatment areas (i.e., treating a plurality of keloids during the same treatment session of the same subject). According to some embodiments, inner elongated needle 220 may be disposed following the treatment session.

According to some embodiments, inner elongated needle 220 may be reusable. Put differently, inner elongated needle 220 may be used in several treatment sessions of several subjects. It is noted that no sterilization of inner elongated needle 220 is required, due to the absence of contact between the inner elongated needle 220 and the treatment areas.

According to some embodiments and as depicted in FIG. 2A, inner elongated needle 220 is unbent. According to some embodiments, inner elongated needle 220 may be at least partially bent. According to some embodiments, inner elongated needle 220 may include or be made of thermally conductive material, such as stainless steels, metals, and the like.

According to some embodiments, inner elongated needle 220 includes a distal portion 202 having a distal opening (not shown), and a proximal portion 204 having a proximal opening (not shown), such that the distal and the proximal openings thereof define a lumen (not shown) therebetween. According to some embodiments, the lumen of inner elongated needle 220 is configured to allow cryogen fluid flow therethrough.

According to some embodiments, proximal portion 204 of the inner elongated needle 220 is configured to be connected directly or indirectly to a cryogen source. According to some embodiments, proximal portion 204 includes circumferentially expanded cone-shaped collars 228 to facilitate connecting and sealing of inner elongated needle 220 to the cryogen source.

According to some embodiments, proximal portion 204 of the inner elongated needle 220 is configured to be connected to a connector (shown in FIG. 3A). According to some embodiments, proximal portion 204 may be detachably or permanently connected to the connector. According to some embodiments, proximal portion 204 may be connected to the connector by screwing, via a screwing segment 224. According to some embodiments, screwing segment 224 may be integrally formed with inner elongated needle 220. According to some embodiments, screwing segment 224 may be couplable to the proximal portion 204 of the inner elongated needle 220 by mechanical pressure, adjusted by tolerance values therebetween.

According to some embodiments, proximal portion 204 may be connected to the connector by various mechanical connection mechanisms, such as snap fit mechanism.

According to some embodiments, inner elongated needle 220 may include 18 G, 19 G, 20 G, 22 G or any other needle size. Each possibly is a separate embodiment. According to some embodiments, length of inner elongated needle 220 may include may be in the range of about 3 cm to about 15 cm. As a non-limiting example, an assemblable cryoneedle may include inner elongated needle 220 having a needle size of 20 G and an outer elongated needle (e.g., outer elongated needle 110) having a needle size of 15 G. According to some embodiments, outer elongated needle 110 may be longer than inner elongated needle 220. As a non-limiting example, outer elongated needle 110 may be about 1 cm longer than inner elongated needle 220.

Reference is now made to FIG. 3A, which schematically illustrates a perspective side view of a connector 330, and FIG. 3B, which schematically illustrates a cross-sectional side view of connector 330, according to some embodiments. According to some embodiments, connector 330 is configured to couple an outer elongated needle (e.g., the previously disclosed outer elongated needle 110) with an inner elongated needle (e.g., the previously disclosed inner elongated needle 220). According to some embodiments, connector 330 may detachably couple the outer and/or inner elongated needles.

According to some embodiments, connector 330 may be configured for a single use in a single subject. According to some embodiments, connector 330 may be used in a single treatment session for treating one treatment area (e.g., a single keloid). According to some embodiments, connector 330 may be used in a single treatment session for treating a plurality of treatment areas (i.e., treating a plurality of keloids during the same treatment session of the same subject). According to some embodiments, connector 330 may be disposed following the single treatment session.

According to some embodiments, connector 330 may be reusable. Put differently, connector 330 may be used in several treatment sessions of several subjects. It is noted that no sterilization of connector 330 is required, due to the absence of contact between connector 330 and the treatment areas.

According to some embodiments, connector 330 includes a distal portion 332 having a distal opening 331, and a proximal portion 334 having a proximal opening 333.

According to some embodiments, distal opening 331 and proximal opening 333 define a lumen 335 therebetween for receiving the outer and inner elongated needles.

According to some embodiments, connector 330 includes one or more flat outer surfaces 336a/336b configured to facilitate coupling and retaining proximal portions of the inner and/or outer elongated needles. According to some embodiments, one or more flat outer surfaces 336a/336b may have various structures, such as flat, circular, oval, rectangular, polygon-like, or any combination thereof. Each possibility is a separate embodiment. According to some embodiments, one or more flat outer surfaces 336a/336b may be at least partially circumferentially expended.

According to some embodiments and as depicted in FIG. 3A, connector 330 includes at least one cone-shaped collar 337 at the proximal portion 334 thereof adapted to receive and retain the inner elongated needle. According to some embodiments, alternatively or additionally, at least one cone-shaped collar 337 may be positioned at the distal portion 332 to facilitate receiving and retaining an assemblable cryoneedle.

According to some embodiments, connector 330 includes a port 338 fluidly connected to lumen 335. According to some embodiments, port 338 may include an outlet port configured to allow exiting the cryogen fluid (typically gas) out of the assemblable cryoneedle. According to some embodiments, port 338 may include an inlet port configured to allow entering the cryogen fluid into the assemblable cryoneedle.

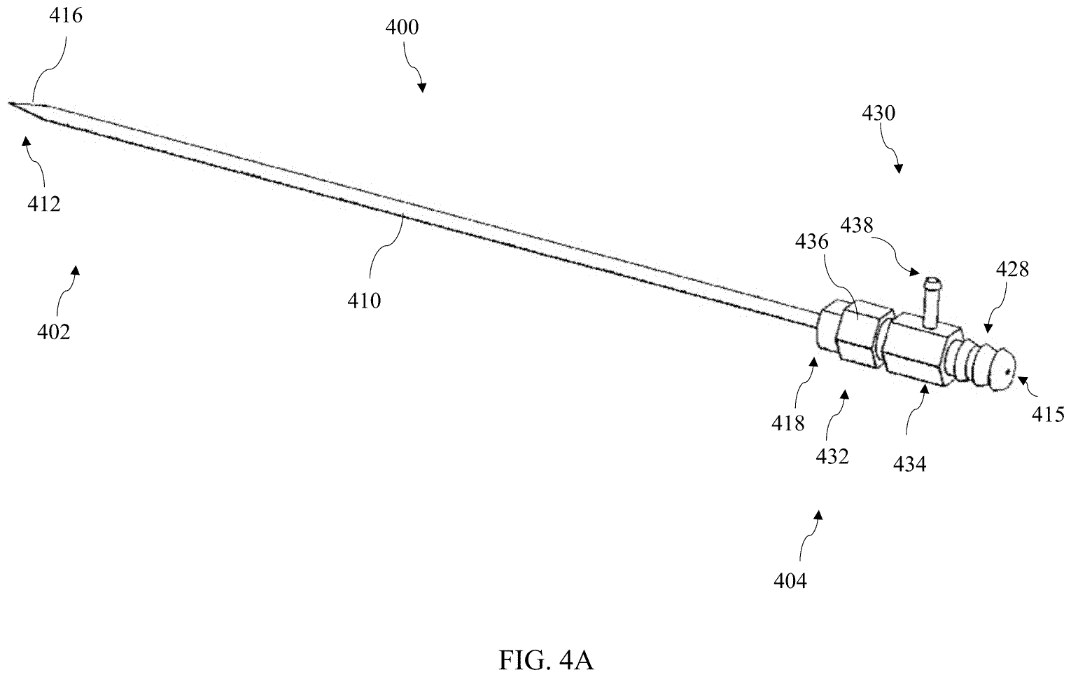

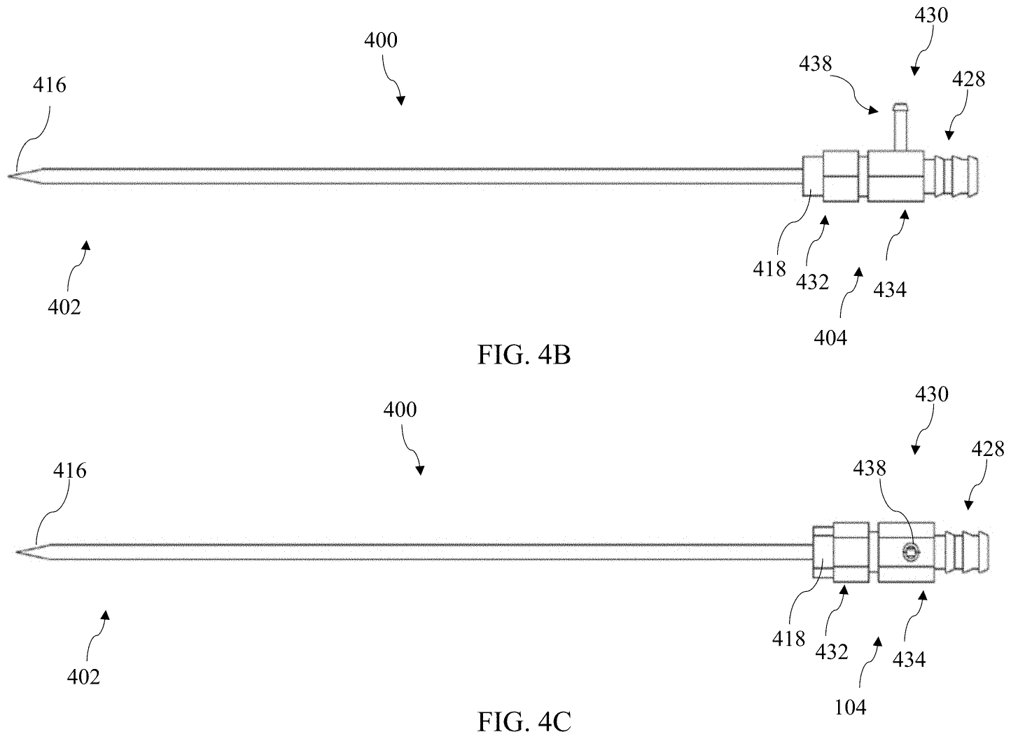

Reference is now made to FIG. 4A, which schematically illustrates a perspective side view of an assemblable cryoneedle 400, FIG. 4B, which schematically illustrates a side view of assemblable cryoneedle 400, and FIG. 4C, which schematically illustrates a top view of assemblable cryoneedle 400 for delivering a cryogen to a treatment area, according to some embodiments. The assemblable cryoneedle 400 includes a distal portion 402 and a proximal portion 404.

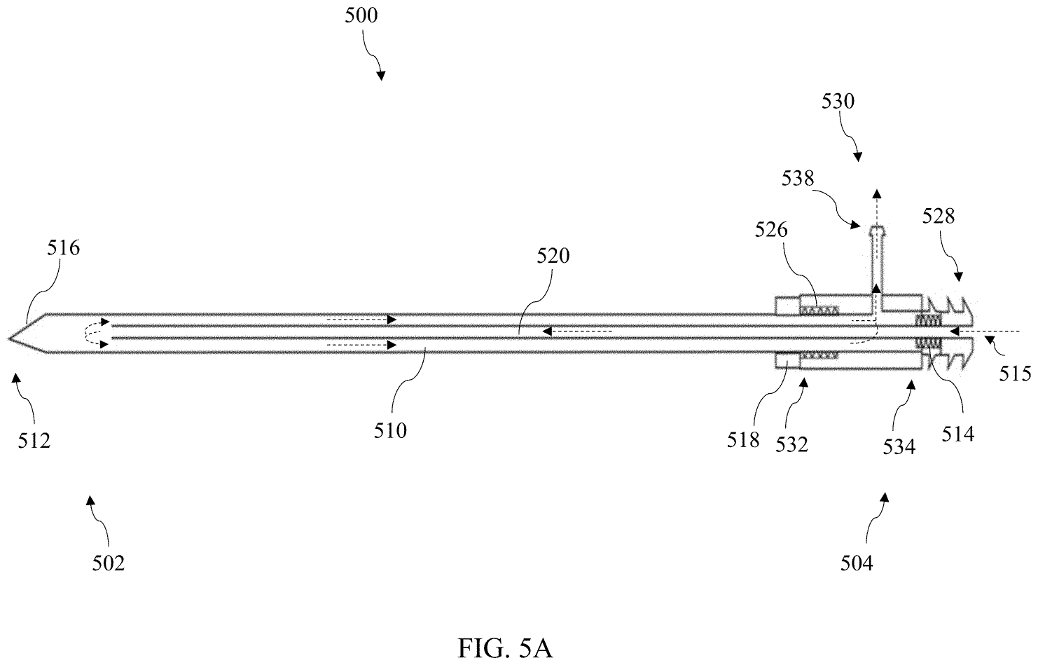

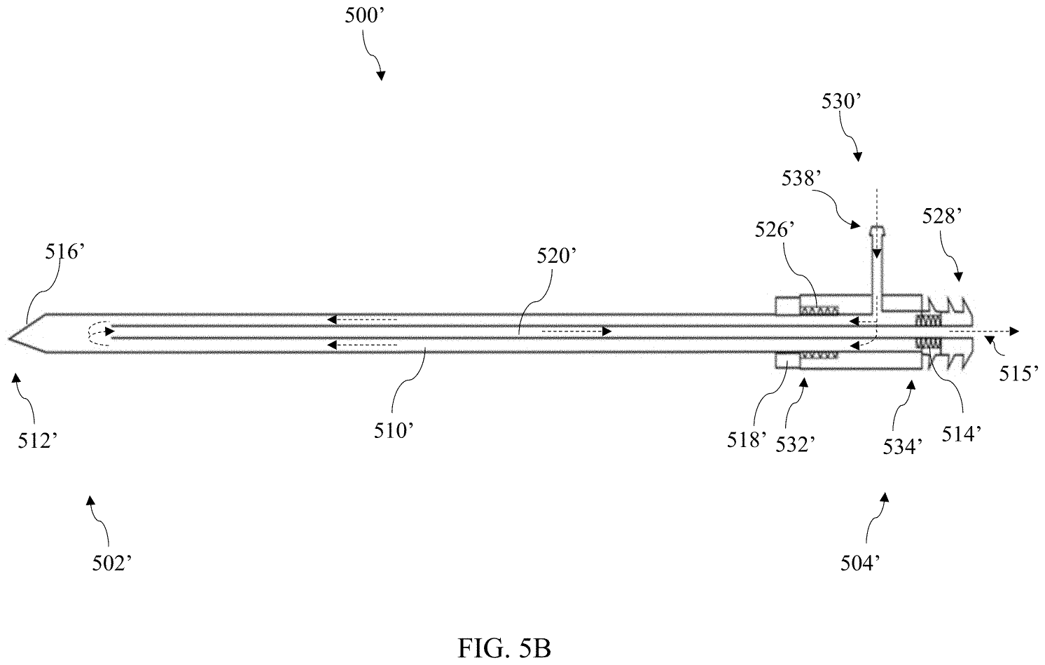

According to some embodiments, assemblable cryoneedle 400 includes an outer elongated needle 410, an inner elongated needle (shown in FIG. 5A hereinbelow) and a connector 430. According to some embodiments and as depicted in FIGS. 4A-C, assemblable cryoneedle 400 is unbent. According to some embodiments, assemblable cryoneedle 400 may be bent, as further elaborated.

According to some embodiments, outer elongated needle 410 includes a sealed distal portion 412 configured to prevent leaking of a cryogen fluid (typically gas) from distal portion 402 of the assemblable cryoneedle 400. Advantageously, and in contrast to currently used welded cryoneedles, the assemblable cryoneedle 400 (i.e., outer elongated needle 410, the inner elongated needle, and/or connector 430) may be manufactured by CNC machining. According to some embodiments, CNC manufacturing process may increase the strength and decrease defects presence at various portions of assemblable cryoneedle 400 (e.g., sealed distal portion 412). Alternatively, the assemblable cryoneedle 400 may be manufactured by other suitable techniques (e.g., laser-assisted manufacturing).

According to some embodiments, sealed distal portion 412 includes an integrally formed sharp tip 416, such as a cutting tip e.g., conventional, tapered, side cutting, K needle or any combination thereof, to facilitate puncturing/penetrating/piercing the treatment area and prevent breakage of the tip. According to some embodiments, the shape of sharp tip 416 may include a conus-like shape, pyramid-like shape, such as triangular, rectangular, diamond, and the like. As a non-limiting example, sharp tip 416 may include triangular shape having 3 cutting edges. According to some embodiments, one of the cutting edges may include concave or convex surface curvatures. In other embodiments, convex and concave surfaces may be flat.

According to some embodiments, sharp tip 416 may be integrally formed with outer elongated needle 410. According to some embodiments, integrally formed sharp tip 416 may be beneficial in different scenarios, e.g., to facilitate penetrating into a hard and stiff tissue, and withdrawing following a prolong cryogenic surgery. Furthermore, integrally formed sharp tip 416 may increase the lifetime of the assemblable cryoneedle by, among other things, decreasing material failure (e.g., cracks propagation, which, in turn, lead to cryogen leakage and, eventually, to material fracture) at the interface area between sharp tip and outer elongated needle.

According to some embodiments, a proximal portion of outer elongated needle 410 may include one or more flat outer surfaces 418 to facilitate coupling and retaining the proximal portion thereof to connector 430. According to some embodiments, one or more flat outer surfaces 418 may have various structures, such as flat, circular, oval, rectangular, polygon-like, or any combination thereof. Each possibility is a separate embodiment. According to some embodiments, one or more flat outer surfaces 418 may be at least partially circumferentially expended.

According to some embodiments, a proximal portion of the inner elongated needle includes an opening 415 (FIG. 4A) configured to allow fluid communication between a cryogen source and assemblable cryoneedle 400, such that cryogen fluid (typically gas) may enter assemblable cryoneedle 400 through opening 415.

Alternatively, according to some embodiments, opening 415 may allow exiting of the cryogen fluid (typically gas). According to some embodiments, opening 415 may be connected to a tube (not shown), such as a flexible tube/vent, to facilitate distancing the cryogen fluid (typically gas) from a treatment area and/or user.

According to some embodiments, a proximal portion of the inner elongated needle (shown in FIG. 5A hereinbelow) further includes circumferentially expanded cone-shaped collars 428 to facilitate connecting and sealing of the inner elongated needle to the cryogen source. According to some embodiments, the inner elongated needle may be directly connected to the cryogen source via cone-shaped collars 428. According to some embodiments, the inner elongated needle may be connected to the cryogen source indirectly, e.g., by using an adapter (not shown), and/or a splitter (as further elaborated herein) and/or an elongated extension tube (not shown). As a non-limiting example, cone-shaped collars 428 may be inserted into a distal end of the elongated extension tube and a proximal end of the elongated extension tube may be connectable to the cryogen source and/or splitter, allowing fluid communication between the cryogen source and opening 415. According to some embodiments, the extending tube may facilitate maneuvering the assemblable cryoneedle 400 during introducing (e.g., puncturing/inserting/piercing) thereof into a treatment area.

According to some embodiments, connector 430 includes a distal portion 432 having a distal opening, and a proximal portion 434 having a proximal opening, defining a lumen therebetween for receiving outer elongated needle 410 and the inner elongated needle (shown in FIG. 5A hereinbelow). According to some embodiments, connector 430 is configured to connect and secure the inner elongated needle to the outer elongated needle 410.

According to some embodiments, connector 430 includes one or more flat outer surfaces 436 configured to facilitate coupling and retaining to the proximal portion of the inner and/or outer needles. According to some embodiments, one or more flat outer surfaces 436 may have various structures, such as flat, circular, oval, rectangular, polygon-like, or any combination thereof. Each possibility is a separate embodiment. According to some embodiments, one or more flat outer surfaces 436 may be at least partially circumferentially expended.

According to some embodiments, one or more flat outer surfaces 436 of connector 430 may be adjusted to match one or more flat outer surfaces 418 of outer elongated needle 410, thus indicating fully secured coupling/assembling thereof. According to some embodiments, the matching may be based, among others, on matching components, such as circumferential teeth, pins, notches, or any other elements.

According to some embodiments, connector 430 may further include an outlet port 438 connected to the lumen, configured to allow exiting the cryogen fluid (typically gas) therethrough. According to some embodiments, outlet port 438 is configured to receive an extension tube (not shown) to distance the exiting cryogen fluid (typically gas) from a subject and/or a user. According to some embodiments, outlet port 438 may be integrally formed with connector 430. According to some embodiments, outlet port 438 may be couplable to connector 430 by mechanical pressure, adjusted by tolerance values therebetween.

According to some embodiments, assemblable cryoneedle 400 is disposable and may be discarded after a single use. According to some embodiments, assemblable cryoneedle 400 is configured for a single use for a single subject. Put differently, assemblable cryoneedle 400 is configured to treating a single subject in a single treatment session. According to some embodiments, assemblable cryoneedle 400 may be used in a single treatment session for treating one treatment area (e.g., a single keloid). According to some embodiments, assemblable cryoneedle 400 may be used in a single treatment session for treating a plurality of treatment areas (i.e., treating a plurality of keloids during the same treatment session of the same subject). According to some embodiments, assemblable cryoneedle 400 may be made of metals and/or stainless steels. According to some embodiments, assemblable cryoneedle 400 may be partially disposable, i.e. include disposable and reusable components. As a non-limiting example, outer elongated needle 410 and inner elongated needle may be disposable, while connector 430 and/or the inner elongated needle may be reusable.

Reference is now made to FIG. 5A, which schematically illustrates a cross-sectional side view of an assemblable cryoneedle 500, according to some embodiments. According to some embodiments, assemblable cryoneedle 500 may be identical, similar or different from the previously disclosed assemblable cryoneedle 400.

According to some embodiments, assemblable cryoneedle 500 includes a distal portion 502 and a proximal portion 504. According to some embodiments and as depicted in FIG. 5A, assemblable cryoneedle 500 is unbent. According to some embodiments, assemblable cryoneedle 500 includes an outer elongated needle 510, an inner elongated needle 520 and a connector 530.

According to some embodiments, a proximal portion of the inner elongated needle 520 may include an opening 515 configured to allow fluid communication between the assemblable cryoneedle 500 and a cryogen source. According to some embodiments, the proximal portion of the inner elongated needle 520 may further include one or more circumferentially expanded cone-shaped collars 528 to facilitate direct or indirect connecting and sealing of the inner elongated needle to the cryogen source.

According to some embodiments, proximal portion of the inner elongated needle 520 may be detachably coupled with connector 530. According to some embodiments, proximal portion of inner elongated needle 520 includes a screwing segment 514 detachably connectable to a proximal portion 534 of connector 530 by screwing. According to some embodiments, proximal portion 504 of assemblable cryoneedle 500, and specifically the proximal portion of inner elongated needle 520, may be detachably coupled with connector 530 by a quick single use connection (not shown). According to some embodiments, the quick connection mechanism may be based on various interlocking mechanisms, such as a snap interlocking mechanism. According to some embodiments, the quick connection mechanism may include a plurality of circumferential teeth (e.g. mounted on inner elongated needle 520) and a plurality of corresponding slots (e.g. formed on connector 530), thereby allowing coupling inner elongated needle 520 with connector 530. According to some embodiments, the plurality of teeth may be asymmetrically positioned and/or differ, e.g., in size, structure, orientation, and the like, such that each of the plurality of teeth may be inserted into each of the plurality of slots, allowing coupling at a preferred orientation only.

According to some embodiments, proximal portion of inner elongated needle 520 may be permanently coupled with connector 530.

According to some embodiments, outer elongated needle 510 includes a sealed distal portion 512. According to some embodiments, sealed distal portion 512 includes a sharp tip 516, such as a cutting tip, e.g., conventional, tapered, side cutting, K needle or any combination thereof, to facilitate puncturing/penetrating a treatment area.

According to some embodiments, a proximal portion of the outer elongated needle 510 may include one or more flat outer surfaces 518 to facilitate coupling and securing thereof with connector 530. According to some embodiments, one or more flat outer surfaces 518 may be at least partially circumferentially expended.

According to some embodiments, connector 530 includes proximal portion 534 having an opening and a distal portion 532 having an opening, thereby defining a lumen therebetween. According to some embodiments, connector 530 further includes an outlet port 538 fluidly connected to the lumen, thereby allowing exiting the cryogen fluid (typically gas) flow therethrough.

According to some embodiments, connector 530 is configured to detachably connect and secure outer elongated needle 510 with inner elongated needle 520 while allowing fluid communication therebetween. According to some embodiments, distal portion 532 of the connector 530 may include a screwing segment 526 on an inner surface thereof geometrically complementary with a screwing segment formed on an outer surface of a proximal portion of the outer elongated needle 510, allowing detachable connecting by screwing. According to some embodiments, proximal portion of outer elongated needle 510 and distal portion 532 of the connector 530 may be connected via a quick connection mechanism, e.g., interlocking mechanism.

According to some embodiments, connector 530 may be permanently coupled with inner elongated needle 520 and/or with outer elongated needle.

According to some embodiments, in operation and as marked by arrows in FIG. 5A, a cryogen fluid (typically gas) enters assemblable cryoneedle 500 through proximal portion 504 thereof. Specifically, the cryogen may flow from opening 515 of inner elongated needle 520 to a sealed distal portion 512 of outer elongated needle 510, then back from the sealed distal portion 512 of the outer elongated needle 510, and out through the outlet port 538 of connector 530. According to some embodiments, outlet port 538 may be connected to a tube (not shown), such as a flexible tube/vent, to facilitate distancing the cryogen fluid from a treatment area and/or user.

Reference is now made to FIG. 5B, which schematically illustrates a cross-sectional side view of an assemblable cryoneedle 500′, according to some embodiments. According to some embodiments, assemblable cryoneedle 500′ may be identical, similar or different from the previously disclosed assemblable cryoneedles 400 and 500.

According to some embodiments, the following components depicted in FIG. 5B 500′, 502′, 504′, 510′, 512′, 514′, 515′, 516′, 518′, 520′, 526′, 528′, 530′, 532′, and 534′ correspond to and may have the same structure and configuration as the previously described components 500, 502, 504, 510, 512, 514, 515, 516, 518, 520, 526, 528, 530, 532, and 534, respectively.

According to some embodiments, in operation and as marked by arrows in FIG. 5B, a cryogen fluid (typically gas) may enter assemblable needle 500′ through an inlet port 538′ of connector 530′ to sealed distal portion 512′ of outer elongated needle 510′, back from a distal portion of inner elongated needle 520′ and out though an opening 515′ at a proximal portion of inner elongated needle 520′.

According to some embodiments, inlet port 538′ of connector 530′ may be directly connected to a cryogen source. According to some embodiments, inlet port 538′ of connector 530′ may be indirectly connected to a cryogen source, e.g., via a splitter, an adapter, an extension tube, and the like.

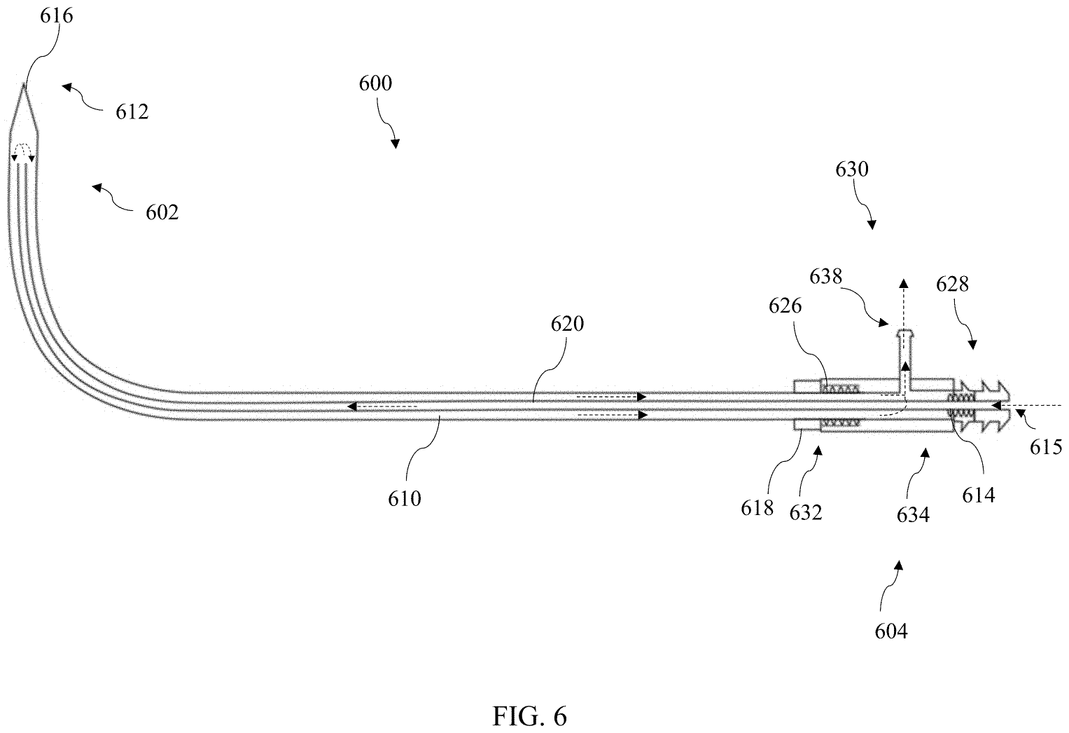

Reference is now made to FIG. 6, which schematically illustrates a cross-sectional side view of an assemblable cryoneedle 600, according to some embodiments. According to some embodiments, assemblable cryoneedle 600 includes a distal portion 602 and a proximal portion 604. The disclosed herein assemblable cryoneedle 600 includes an outer elongated needle 610, an inner elongated needle 620 and a connector 630.

According to some embodiments, outer elongated needle 610 includes a sealed distal portion 612. According to some embodiments, sealed distal portion 612 may include a sharp tip 616 to facilitate puncturing/penetrating a treatment area. According to some embodiments, sharp tip 616 may include a cutting tip, e.g., conventional, tapered, side cutting, K needle or any combination thereof. According to some embodiments, the outer elongated needle 610 may be manufactured by CNC machining, such that the sharp tip 616 is integrally formed with the outer elongated needle 610. According to some embodiments, integrally formed sharp tip 616 may increase the lifetime of the assemblable cryoneedle 600, including, among other things, decreasing material failure (e.g., cracks propagation, which, in turn, lead to cryogen leakage and, eventually, to material fracture) at the interface area between sharp tip 616 and outer elongated needle 610.

According to some embodiments, a proximal portion of the outer elongated needle 610 may include one or more flat outer surfaces 618 to facilitate coupling and securing thereof with connector 630. According to some embodiments, one or more flat outer surfaces 618 may be at least partially circumferentially expending.

According to some embodiments, a proximal portion of inner elongated needle 620 may include one or more circumferentially expanding cone-shaped collars 628 to facilitate direct or indirect connecting and sealing of the inner elongated needle to the cryogen source.

According to some embodiments, connector 630 includes a distal portion 632 having a distal opening, and a proximal portion 634 having a proximal opening, defining a lumen therebetween. According to some embodiments, connector 630 is configured to connect and secure the inner elongated needle 620 to the outer elongated needle 610. In particular, according to some embodiments, distal portion 632 of connector 630 is connectable to proximal portion of the outer elongated needle 610, and proximal portion 634 of the connector 630 is connectable to the proximal portion of the inner elongated needle 620.

According to some embodiments, distal portion 632 of the connector 630 may include a screwing segment 626 on an inner surface thereof geometrically complementary with a screwing segment formed on an outer surface of the proximal portion of the outer elongated needle 610, allowing detachable connecting by screwing. According to some embodiments, proximal portion 634 of connector 630 may be detachably connected with the proximal portion of the inner elongated needle 620, e.g., by screwing via a screwing segment 614 of inner elongated needle 620.

According to some embodiments, connector 630 may be detachably connected with the inner elongated needle 620 and/or outer elongated needle 610 via a quick connection mechanism, e.g., interlocking mechanism.

According to some embodiments, assemblable cryoneedle 600 may be at least partially bent. According to some embodiments and as depicted in FIG. 6 inner elongated needle 620, outer elongated needle 610 and distal portion of assemblable cryoneedle 600 are bent. According to some embodiments, distal portion 602 of assemblable cryoneedle 600 may be bent while inner elongated needle 620 may be unbent. It is understood by one skilled in the art that although assemblable cryoneedle 600 is bent, the position of the inner elongated needle 620 respectively to the outer elongated needle 610 remains substantially constant, for providing continuous cryogen flow therethrough.

According to some embodiments, outer elongated needle 610 and inner elongated needle 620 and are concentric. According to some embodiments, outer elongated needle 610 and/or inner elongated needle 620 may typically include a circular cross-section. According to some embodiments, outer elongated needle 610 and/or inner elongated needle 620 may include various cross-sectional shapes, such as oval, triangular, rectangular, polygon-like, and the like. According to some embodiments, the outer elongated needle 610 may include 14 G, 16 G, 18 G, 20 G, 22 G or any other needle size. Each possibly is a separate embodiment. According to some embodiments, inner elongated needle 620 may include 18 G, 20 G, 22 G or any other needle size. Each possibly is a separate embodiment. As a non-limiting example, inner elongated needle 620 may include 20 G and outer elongated needle 610 may include 15 G needle sizes. According to some embodiments, outer elongated needle 610 may be longer than inner elongated needle 620. As a non-limiting example, outer elongated needle 610 may be about 1 cm longer than inner elongated needle 620.

According to some embodiments, in operation and as marked by arrows in FIG. 6, a cryogen fluid (typically gas) enters assemblable cryoneedle 600 through proximal portion 604 thereof. Specifically, the cryogen may flow from opening 615 of inner elongated needle 620 to sealed distal portion 612 of outer elongated needle 610, then back from sealed distal portion 612 of outer elongated needle 610, and out through outlet port 638 of connector 630.

Alternatively, in operation, the cryogen fluid may enter assemblable cryoneedle 600 through proximal portion 604 thereof, and specifically via outlet port 638 of connector 630. Put differently, according to some embodiments outlet port 638 may be adapted to receive cryogen flow into assemblable cryoneedle 600 (i.e., serve as an inlet port). According to some embodiments, then the cryogen may flow to sealed distal portion 612 of outer elongated needle 610, back from a distal portion of inner elongated needle 620 and out though opening 615 at proximal portion of inner elongated needle 620.

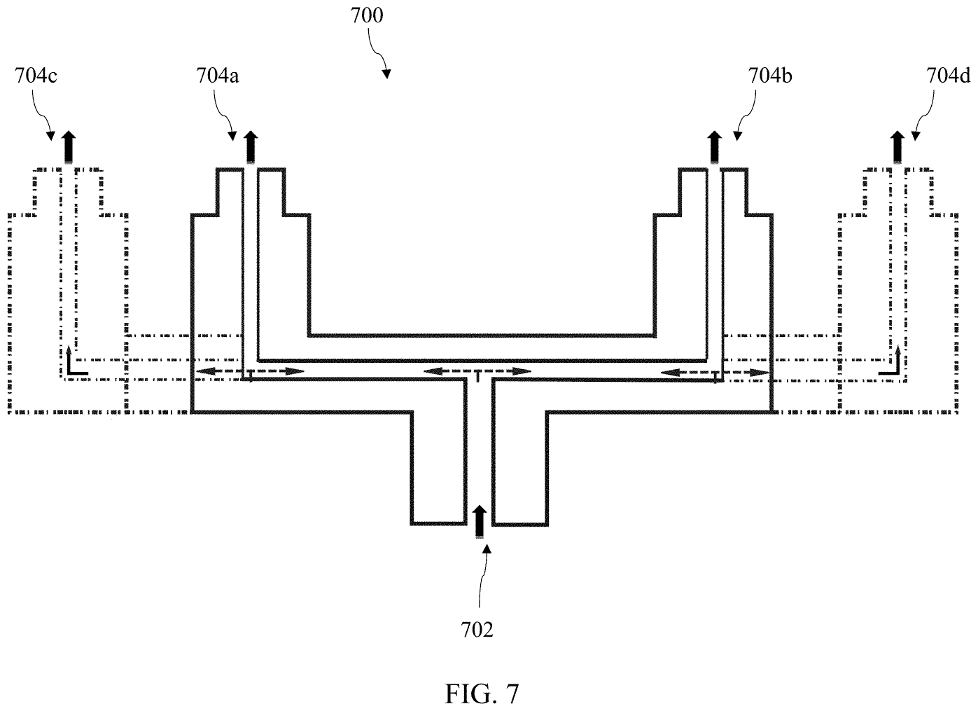

Reference is now made to FIG. 7, which schematically illustrates a cross-sectional top view of a splitter 700, according to some embodiments. According to some embodiments, splitter 700 facilitates simultaneously connecting a plurality of assemblable cryoneedles to a single cryogen source while preserving the working pressure to each of the plurality of assemblable cryoneedles. According to some embodiments, the working pressure of each of the plurality of assemblable cryoneedles is in the range of about 10-12 Psi. According to some embodiments and as depicted in FIG. 7, splitter 700 may include two outlet ports 704a, 704b, and optionally may include additional outlet ports 704c and/or 704d, allowing simultaneously connecting one, two, three or four assemblable cryoneedles to the cryogen source. According to some embodiments, splitter 700 may include 2, 3, 4, 5, 6, 7, 8, 9, 10 or more outlet ports. Each possibility is a separate embodiment. According to some embodiments, splitter 700 may include a different number of outlet ports.

According to some embodiments, each of outlet ports 704a, 704b, 704c, and 704d, and/or inlet port 702 may include circular, cylindrical, square, rectangular, polygonal or polygon-like lateral cross-section. According to some embodiments and as depicted in FIG. 7, the diameter of inlet port 702 is greater than the diameter of each of outlet ports 704a, 704b, 704c, and 704d to facilitate providing and maintaining the desired working pressure.

According to some embodiments, in operation and as marked by arrows in FIG. 7, a cryogen fluid (typically gas) enters inlet port 702, flows across a fluid passage extending through splitter 700 from inlet port 702 to each of outlet ports 704a, 704b, 704c, and 704d and exists therefrom.

According to some embodiments, splitter 700 may include pressure regulators (not depicted), such as valves, to facilitate stabilizing the cryogen fluid pressure and/or manipulating the cryogen fluid flow therethrough.

According to some embodiments, splitter 700 may be directly connected to the cryogen source via an inlet port 702. According to some embodiments, inlet port 702 may be indirectly connected to the cryogen source, e.g., via a connector, adapter, extension tube, and the like.

According to some embodiments, splitter 700 may be rigid and/or semi-rigid. According to some embodiments, splitter 700 may be manufactured from an insulating material, such as polymeric material.

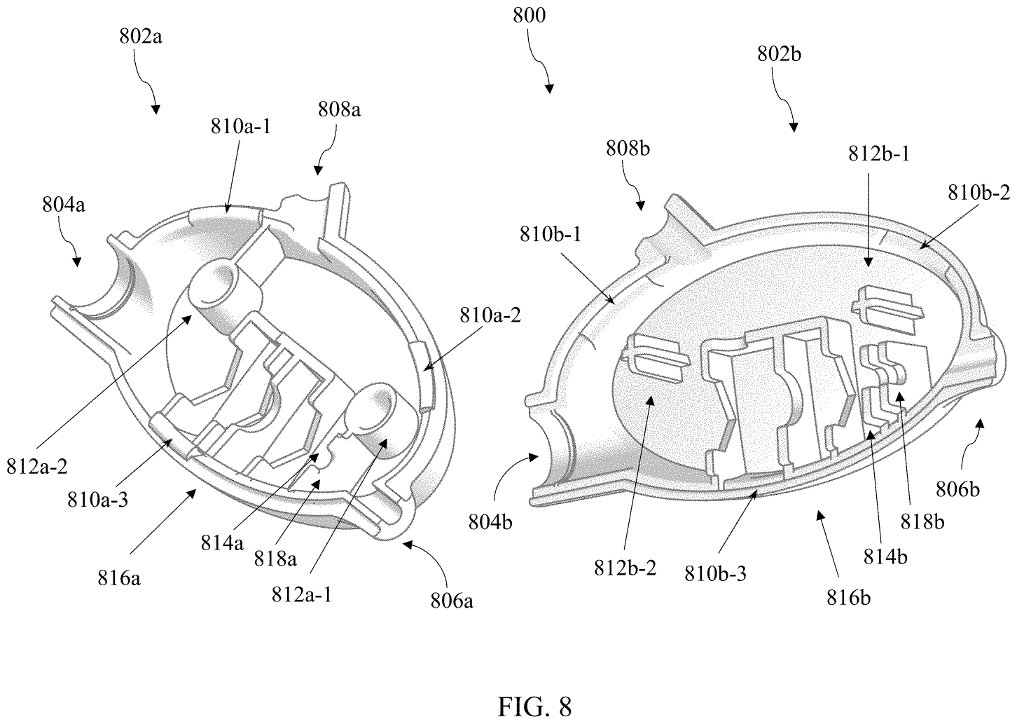

Reference is now made to FIG. 8, which schematically illustrates a side view of one or more thermally insulating gripping elements 800, according to some embodiments. According to some embodiments and as depicted in FIG. 8, one or more thermally insulating gripping elements 800 includes a first gripping element 802a and a second gripping element 802b, configured to facilitate gripping of an assemblable cryoneedle by a user. According to some embodiments, first gripping element 802a is geometrically complementary with second gripping element 802b.

According to some embodiments, one or more thermally insulating gripping elements 800 may include textured outer surfaces (not depicted) to facilitate the grip and minimize dislocating of the assemblable cryoneedle during a medical procedure.

According to some embodiments, one or more thermally insulating gripping elements 800 (e.g., first gripping element 802a and second gripping element 802b) are configured to cover a connector and/or at least a section of an outer/inner elongated needle, thereby facilitating gripping of the assemblable cryoneedle by the user during introduction thereof into the treatment area. According to some embodiments, one or more thermally insulating gripping elements 800 configured for preventing the user a frostbite injury due to gripping the assemblable cryoneedle during and following the cryo-treatment, thus advantageously allowing safely performing cryo-treatments for minutes to hours. According to some embodiments, one or more thermally insulating gripping elements 800 are configured to snap fit and cover the connector, providing a comfortable grip of the assemblable cryoneedle. Scenarios wherein it may be beneficial include facilitating positioning the assemblable cryoneedle at a specific orientation/angle for approaching/puncturing a treatment area, and/or when a prolonged medical procedure is performed, and/or during withdrawal of the assemblable cryoneedle.

According to some embodiments, one or more thermally insulating gripping elements 800 may be made of any suitable insulating materials. According to some embodiments, one or more thermally insulating gripping elements 800 may be made of or include plastic materials, such as polycarbonate. According to some embodiments, manufacturing techniques of the one or more thermally insulating gripping elements 800 may include molding, e.g., pressure molding. According to some embodiments, one or more thermally insulating gripping elements 800 may be manufactured by 3D-printing. According to some embodiments, one or more thermally insulating gripping elements 800 may be constructed using machine processing (machining).

According to some embodiments, first gripping element 802a may include a first opening 804a adapted to the geometry (i.e. dimensions and shape) of a cryogen source and/or a tube/adapter configured to connect the assemblable cryoneedle to the cryogen source. According to some embodiments, opening 804a is configured to cover a proximal portion of the assemblable cryoneedle (e.g., a proximal portion of the connector and/or a proximal portion of an inner elongated needle). According to some embodiments, the second gripping element 802b may include a first opening 804b geometrically complementary with first opening 804a of first gripping element 802a.

According to some embodiments, first gripping element 802a may include a second opening 806a configured to cover the outer elongated needle and/or a distal portion of the connector. According to some embodiments, second gripping element 802b may include a second opening 806b geometrically complementary with the second opening 806a of the first gripping element 802a.

According to some embodiments, first gripping element 802a may include a third opening 808a configured to cover an outlet and/or an inlet port of the connector. According to some embodiments, the third opening 808a is configured to cover an extension tube connected to the outlet/inlet port of the connector. According to some embodiments, second gripping element 802b may include a third opening 808b geometrically complementary with third opening 808a of the first gripping element 802a.

According to some embodiments, the first gripping element 802a may include one or more circumferential locking elements 810a-1/810a-2/810a-3 adapted to receive one or more geometrically complementary circumferential locking elements 810b-1/810b-2/810b-3 of the second gripping element 802b, configured for locking and securing the first gripping element 802a with the second gripping element 802b. According to some embodiments, first gripping element 802a and second gripping element 802b may be locked and secured via a quick-locking mechanism, such as a snap fit mechanism.

According to some embodiments, first gripping element 802a includes one or more inner locking elements 812a-1/812a-2 adapted to receive one or more geometrically complementary inner locking elements 812b-1/812b-2, respectively, of the second gripping element 802b. As a non-limiting example, one or more inner locking elements 812a-1/812a-2 include cylindrical elements configured to receive the respective inner locking elements 812b-1/512b-2, thereby facilitating locking and securing the one or more gripping elements 800. According to some embodiments, inner locking elements 812b-1/812b-2 include protruding elements configured to be inserted into the respective inner locking elements 812a-1/812a-2.

According to some embodiments, one or more gripping elements 800 include first protruding elements 816a/816b adapted to receive the connector and/or a portion of the assemblable cryoneedle. According to some embodiments, first protruding elements 816a/816b are geometrically complementary with one or more flat surfaces of the connector, thereby allowing covering of the connected at a predefined orientation and facilitating locking one or more gripping elements 800 thereon.

According to some embodiments, first gripping element 802a includes one or more second protruding elements 818a adapted to receive a proximal portion of the outer elongated needle. According to some embodiments, one or more second protruding elements 818a are configured to facilitate retaining and securing the outer elongated needle at a predefined position when covered by one or more thermally insulating gripping elements 800, and specifically during penetration and withdrawal from the treatment area. According to some embodiments, one or more second protruding elements 818a are geometrically complementary with one or more second protruding elements 818b of second gripping element 802b. According to some embodiments and as depicted in FIG. 8, one or more second protruding elements 818a includes one second protruding element 818a adapted for insertion between two second protruding elements 818b. According to some embodiments, the number and shape of one or more second protruding elements 818a/818b may vary.

According to some embodiments, first gripping element 802a includes a groove 814a between first protruding elements 816a and of one or more second protruding elements 818a. According to some embodiments, groove 814a is geometrically complementary with a groove 814b positioned between first protruding elements 816b and one or more second protruding elements 818b of second gripping element 802b. According to some embodiments, grooves 814a/814b are adapted to receive one or more flat outer surfaces of the outer elongated needle (e.g., one or more flat outer surfaces 418 depicted in FIG. 4A) and are configured to further facilitate strengthening and retaining the proximal portion of the outer elongated needle to the connector during approaching/puncturing/piercing the treatment area (e.g., preventing bending or cracking of the assemblable cryoneedle) and during the withdrawal of the assemblable cryoneedle (e.g., preventing the detachment of the outer elongation tube from the connector) at the end of the cryo-treatment.

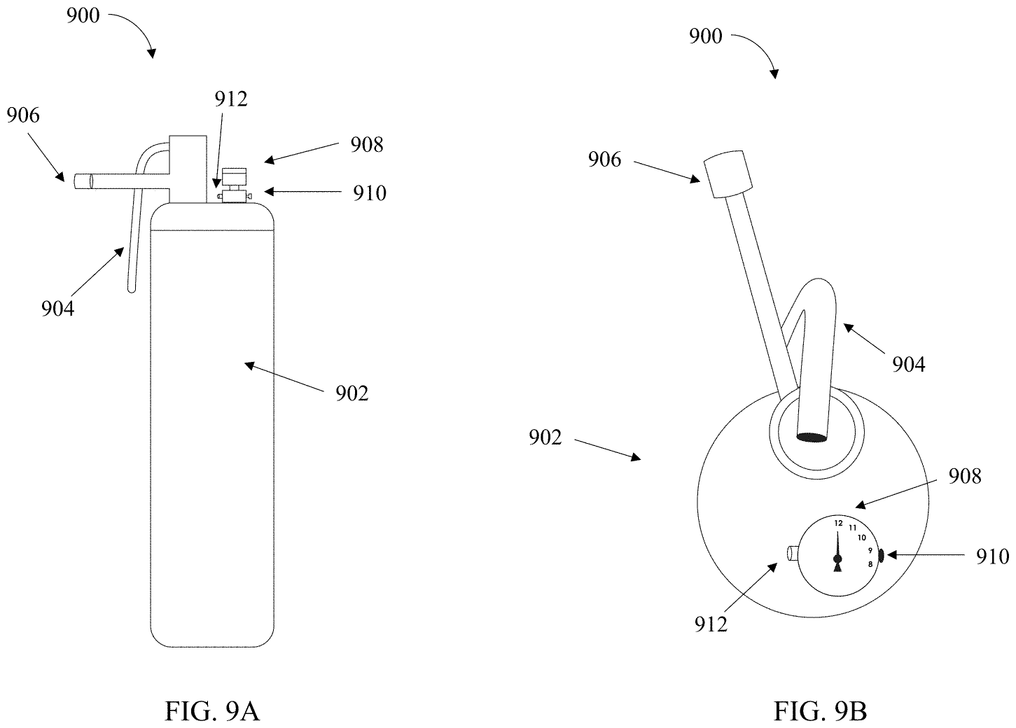

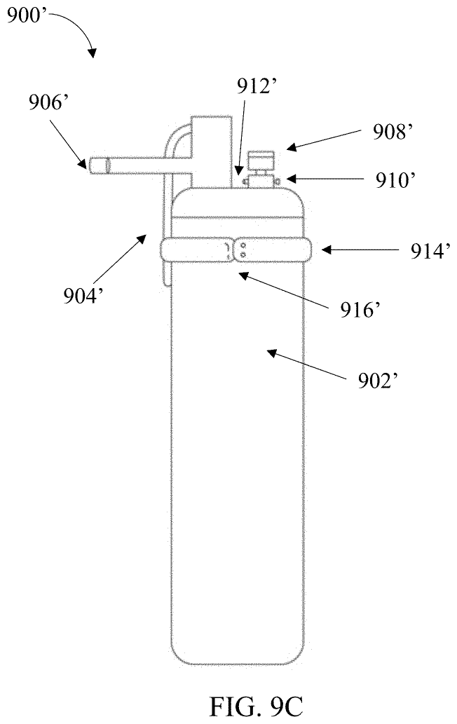

Reference is now made to FIG. 9A, which schematically depicts a side view of a cryogen source system 900 for supplying a cryogen fluid, and FIG. 9B, which schematically depicts a top view of cryogen source system 900, according to some embodiments. According to some embodiments, cryogen source system 900 includes a cryogen source container 902 configured to store and provide the cryogen fluid (typically gas). According to some embodiments, cryogen source container 902 includes an opening 906 configured to be opened (i.e. allowing fluid flow therethrough) or closed (i.e. preventing fluid flow therethrough) according to the position of a trigger 904. According to some embodiments, the position of trigger 904 may be manually controlled by the user.

According to some embodiments, system 900 further includes a pressure gauge 908, configured to display the current pressure, and an adjustable pressure regulator 910 configured for manually regulating the pressure of the supplied cryogen fluid. Thus, advantageously allowing performing continuous and uninterrupted cryo-treatments of scars and keloids for minutes to hours, in contrast to the commonly performed cryo-treatments, such as dermatological cryo-treatments which typically last several seconds. According to some embodiments, system 900 includes a safety valve 912 configured for venting out exceeding internal pressure (i.e. above a predefined threshold pressure) from cryogen source container 902, thereby increasing the safety of the user and the subject.

According to some embodiments, cryogen source container 902 is a portable container. As a non-limiting example, cryogen source container 902 may include capacity of about 500 ml, about 500-1000 ml or any other portable capacity of a cryogen fluid (typically gas). It may be understood by one skilled in the art that the portable container enables executing a cryo-treatment for about 45 to 60 minutes, and thereafter, i.e., upon emptying the portable container, it must be exchanged with another filled-up portable container. According to some embodiments, cryogen source container 902 may be a stationary container, such as a storage tank. According to some embodiments, the stationary container may have a capacity of about 5, 10, 25, 50 liters or more. According to some embodiments, the stationary container may include any other capacity. Each possibility is a separate embodiment. It may be understood by one skilled in the art that such a stationary container enables executing longer cryo-treatments, i.e., lasting more than about 45 to about 60 minutes (e.g., lasting about 1 hour, about 2 hours, about 3 hours or more hours), without exchanging the stationary container. According to some embodiments, the stationary container may be functionally associated with adjustable pressure regulator 910.

According to some embodiments, cryogen source container 902 may be positioned in the vicinity of the subject (e.g., a portable container, positioned by the user). According to some embodiments, cryogen source container 902 may be positioned at various locations, such as in a designated area in a treatment room, outside the room, and the like.

According to some embodiments, cryogen source container 902 stores a cryogen fluid (typically gas), such as, but not limited to, liquid nitrogen. The prior art cryogen source containers are typically filled liquid nitrogen providing pressure of about 8-12 Psi, according to the standards of manufacturing. However, continuous operation of the disclosed herein assemblable cryoneedle requires an approximate pressure value of at least 10 Psi, due to high resistance therein. It is noted that prior art cryogen source containers may be filled with cryogen fluid (typically gas) providing pressure of about 30 Psi or more, which are not applicable in dermatological medical procedures due to safety reasons. Advantageously, the disclosed herein adjustable safety pressure regulator 910 allows the user to regulate the pressure of the supplied cryogen fluid in the desired range—above about 10 Psi and up to about 12 Psi—ensuring continuous, uninterrupted, and substantially constant operating pressure, enabling safe supply of the cryogen fluid and consistent and accurate freezing of the treatment area. According to some embodiments, any internal pressure above about 12 Psi is vented out by the safety valve 912.

Reference is now made to FIG. 9C, which schematically depicts a side view of a cryogen source system 900′ for supplying a cryogen fluid (typically gas), according to some embodiments.

According to some embodiments, the following components depicted in FIG. 9B 902′, 904′, 906′, 908′, 910′, and 912′ correspond to and may have the same structure and configuration as the previously described components 902, 904, 906, 908, 910, and 912, respectively.

According to some embodiments, cryogen source system 900′ includes a non-elastic fastener 914′, configured to maintain continuous supply of the cryogen during minutes to hours without a need of a user holding trigger 904′ of cryogen source container 902′ in an open position, thus advantageously preventing the user a frostbite injury due to the prolonged and continuous gripping the cryogen source container 902′. According to some embodiments, fastener 914′ includes a quick release mechanism 916′ to immediately cease the cryogen supply. According to some embodiments, quick release mechanism 916′ may include an interlocking mechanism, snap fit mechanism, and the like.

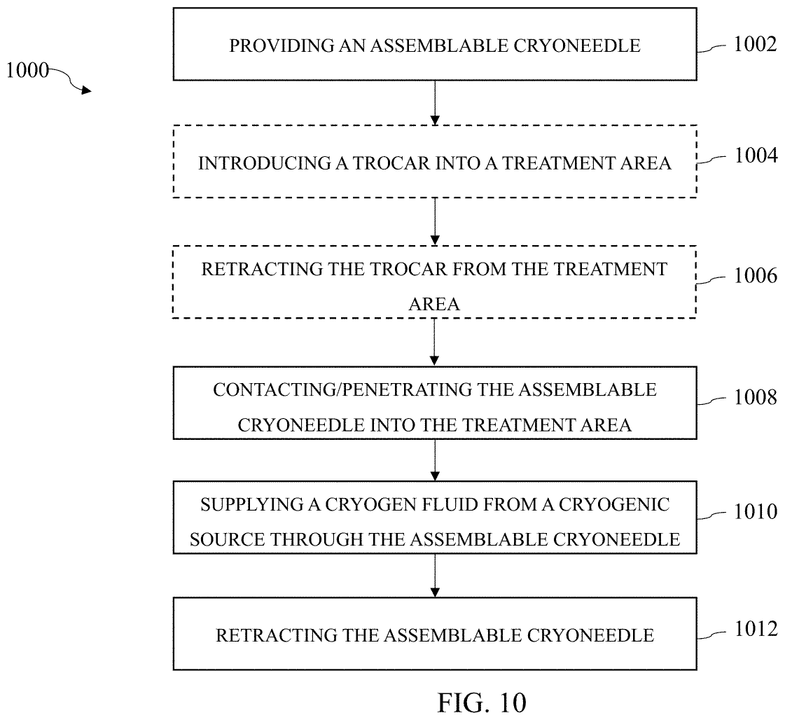

Reference is now made to FIG. 10, which is a flow chart 1000 of the herein disclosed method for performing a medical procedure using an assemblable cryoneedle, according to some embodiments. It is understood by one of ordinary skill in the art that the steps as outlined below may not necessarily be carried out in the indicated order.

In step 1002, an assemblable cryoneedle may be provided for performing the medical procedure.

According to some embodiments, the medical procedure may include cryotherapy, which is also known as cryosurgery and cryoablation. According to some embodiments, the medical procedure may be performed approximately during minutes to hours, or any other duration. According to some embodiments, the medical procedure may be performed repeatedly/sequentially to improve long-term results. As a non-limiting example, the medical procedure may be repeated every about 12 to about 24 weeks.

According to some embodiments, the provided assemblable cryoneedle is configured for a single use for a single subject. According to some embodiments, the provided assemblable cryoneedle may include single use components (e.g., an outer elongated needle) and multi-use components (e.g., connector and/or inner elongated needle).

According to some embodiments, a kit for cryo-treatment may be provided. According to some embodiments, the kit may include one or more assemblable cryoneedles. According to some embodiments, each of the one or more assemblable cryoneedles may include different dimensions, e.g., different lengths, to facilitate approaching various types of keloids. According to some embodiments, the kit may include one or more thermally insulating gripping elements configured to facilitate gripping of the one or more assemblable cryoneedles. According to some embodiments, the kit may include a non-elastic fastener configured to maintain continuous supply of a cryogen fluid without a need of a user holding a trigger of a cryogen source in an open position, thus preventing frostbite injuries. According to some embodiments, the fastener may include a quick release mode to immediately cease the cryogen supply.

According to some embodiments, the kit may include a splitter for facilitating simultaneous connecting of a plurality of cryoneedles to a single cryogen source. According to some embodiments, the kit may include a trocar configured for introducing into the treatment area prior to inserting the cryoneedle, to facilitate the cryoneedle insertion. According to some embodiments, the trocar preferably includes a sharp cutting sealed distal tip. According to some embodiments, the outer diameter of the outer elongated needle is larger than the outer diameter of the trocar.

In step 1004, which is an optional step according to some embodiments, a trocar may be introduced into a treatment area of a subject. According to some embodiments, the treatment area may include tissue, such as abnormal skin tissue (e.g. scars, keloids). According to some embodiments, the treatment area may include internal scar tissue.

Different scenarios wherein in might be beneficial to introduce the trocar prior to introducing the assemblable cryoneedle into the treatment area may include treatment areas having high puncture resistance requiring application of forces, and/or when puncturing is performed using an assemblable cryoneedle with a blunt distal portion.

According to some embodiments, visualizing/imaging techniques may be implemented during introduction of the trocar into the treatment area. According to some embodiments, visualizing/imaging techniques may include, among others, ultrasound or CT guided insertion of the trocar.

In step 1006, which is an optional step according to some embodiments, the trocar may be retracted from the treatment area of the subject. According to some embodiments, retracting the trocar may facilitate performing a step 1008.

In step 1008, the assemblable cryoneedle may be penetrated/contacted into the treatment area of the subject.

According to some embodiments, the assemblable cryoneedle may be positioned in the vicinity of the treatment area. According to some embodiments, the assemblable cryoneedle may puncture and penetrate the treatment area. According to some embodiments, the assemblable cryoneedle may puncture the treatment area such that the interface area between the assemblable cryoneedle and the treatment area is maximized, to facilitate cooling/freezing thereof. According to some embodiments, a direct contact between the assemblable cryoneedle and the treatment area may increase cooling/freezing efficiency, specifically during procedures lasting several hours.

According to some embodiments, step 1008 may include rotating the assemblable cryoneedle to facilitate insertion into the treatment area. According to some embodiments, step 1008 may include linear and/or rotational movement. According to some embodiments, step 1008 may include performing repetitive circular back-and-forth motion to facilitate penetrating into the treatment area. According to some embodiments, step 1008 may include repetitive rotating of the assemblable cryoneedle in circular back-and-forth motion along an angle-limited circular path, e.g., by partially rotating the assemblable cryoneedle. Put differently, the repetitive circular motion may include angle limited clockwise and counterclockwise rotational motions.

According to some embodiments, the assemblable cryoneedle may be inserted clockwise into the treatment area. Alternatively, the assemblable cryoneedle may be inserted counterclockwise into the treatment area. It may be understood by one skilled in the art that the direction of the rotation depends on the screwing connection, if present. According to some embodiments, no rotation of the assemblable cryoneedle is required during the insertion.

According to some embodiments, visualizing/imaging techniques may be implemented during the insertion of the assemblable cryoneedle into the treatment area. According to some embodiments, visualizing/imaging techniques may include, among others, ultrasound or CT guided insertion of the assemblable cryoneedle.

In step 1010, a cryogen fluid may be supplied from a cryogenic source through the assemblable cryoneedle. According to some embodiments, the cryogen source may supply a gas, such as liquid nitrogen.

According to some embodiments, the cryogen source may be equipped with an adjustable pressure regulator. Different scenarios wherein a presence of the adjustable pressure regulator may be beneficial include maintaining the pressure of the supplied cryogen fluid above about 10 Psi, for providing a sufficient and consistent cryogen flow through the assemblable cryoneedle and facilitating heat exchange between the assemblable cryoneedle and the treatment area. It is understood by one skilled in the art that the adjustable pressure regulator is also beneficial for preventing pressure elevation for safety reasons (i.e. safety of the subject and/or the user).

According to some embodiments, the cryogen fluid may flow from a proximal portion to a distal portion of an inner elongated needle of the assemblable cryoneedle, then back from a sealed distal portion of an outer elongated needle, and out through an outlet port of a connector of the assemblable cryoneedle. Alternatively, the cryogen fluid may be supplied through an inlet port of the connector and flow from the proximal to the distal portion of the outer elongated needle, back from the sealed distal portion of the outer elongated needle into the distal portion of the inner elongated needle, and out through the proximal portion of the inner elongated needle.

According to some embodiments, the cryogen fluid may be continuously supplied during minutes to hours, for example, about 5 minutes to 1 hour, about 15 minutes to 2 hours, etc., or any other duration. Each possibility is a separate embodiment.

In step 1012, the assemblable cryoneedle may be retracted/withdrawn from the treatment area. According to some embodiments, retracting the assemblable cryoneedle may be performed in the same direction as the inserting of the assemblable cryoneedle.

According to some embodiments, step 1012 may include rotating the assemblable cryoneedle to facilitate withdrawing thereof from the treatment area. According to some embodiments, step 1012 may include linear and/or rotational movement. According to some embodiments, step 1012 may include performing repetitive circular back-and-forth motion.

According to some embodiments, retracting the assemblable cryoneedle may be performed by rotating the assemblable cryoneedle clockwise in order to break a connection, e.g., screwing connection, between the outer elongated needle and the connector, and render the assemblable cryoneedle a single use assemblable cryoneedle.

What is claimed is:

1. An assemblable cryoneedle comprising:
an outer elongated needle, configured to be inserted into a treatment area of a subject, the outer elongated needle comprising a sealed distal portion and a proximal portion comprising an opening;
an inner elongated needle, configured to be inserted into the outer elongated needle, the inner elongated needle comprising:
a proximal portion, having a proximal opening and configured to be connected to a cryogen source, and
a distal portion, having a distal opening, wherein the proximal and distal openings of the inner elongated needle define a lumen there between; and
a connector comprising:
a distal portion having a distal opening;
a proximal portion having a proximal opening, wherein the connector's proximal and distal openings define a lumen there between; and
an outlet port fluidly connected to the lumen,
the connector is configured to detachably couple the outer elongated needle with the inner elongated needle,
wherein the connector is configured to facilitate flow of a cryogen from the proximal portion to the distal portion of the inner elongated needle, back from the sealed distal portion of the outer elongated needle, and out through the outlet port of the connector.

2. An assemblable cryoneedle comprising:
an outer elongated needle, configured to be inserted into a treatment area of a subject, the outer elongated needle comprising a sealed distal portion and a proximal portion comprising an opening;
an inner elongated needle, configured to be inserted into the outer elongated needle, the inner elongated needle comprising:
a proximal portion, having a proximal opening, and
a distal portion, having a distal opening, wherein the proximal and distal openings of the inner elongated needle define a lumen there between; and
a connector comprising:
a distal portion having a distal opening;
a proximal portion having a proximal opening, wherein the connector's proximal and distal openings define a lumen there between; and
an inlet port fluidly connected to the lumen,
the connector is configured to detachably couple the outer elongated needle with the inner elongated needle,
wherein the connector is configured to facilitate flow of a cryogen from the proximal portion to the distal portion of the outer elongated needle, back from the sealed distal portion of the outer elongated needle into the distal portion of the inner elongated needle, and out through the proximal portion of the inner elongated tube.

3. The cryoneedle of claim 1, wherein the proximal portion of the outer elongated needle is connectable to the distal portion of the connector, and the proximal portion of the inner elongated needle is connectable to the proximal portion of the connector.

4. The cryoneedle of claim 1, wherein the outer elongated needle, the inner elongated needle and/or the connector are manufactured by CNC machining.

5. The cryoneedle of claim 1, wherein the sealed distal portion of the outer elongated needle further comprising a sharp tip configured to penetrate the treatment area.

6. The cryoneedle of claim 1, wherein at least a portion of the outer elongated needle and the inner elongated needle are bent, and wherein a fluid flow circulation capacity is maintained between an outer surface of the inner elongated needle and an inner surface of the outer elongated needle and inside the inner elongated needle.

7. The cryoneedle of claim 1, wherein the proximal portion of the inner elongated needle and/or the inlet/outlet port of the connector is configured to be connected directly or indirectly to a cryogen source via a splitter, the splitter facilitates concomitantly connecting a plurality of cryoneedles to a single cryogen source.

8. The cryoneedle of claim 1, wherein the proximal portion of the outer elongated needle is detachably connectable to the distal portion of the connector by screwing.

9. The cryoneedle of claim 1, wherein the proximal portion of the inner elongated needle is detachably connectable to the proximal portion of the connector by screwing.

10. The cryoneedle of claim 1, wherein at least a portion of the proximal portion of the outer elongated needle is circumferentially expanded (comprising a one or more flat outer surfaces) to facilitate coupling and retaining the proximal portion of the outer elongated needle to the connector.

11. The cryoneedle of claim 1, wherein the proximal portion of the inner elongated needle further comprising one or more circumferentially expanded cone-shaped collars, to facilitate direct or indirect connection and sealing of the inner elongated needle to the cryogen source.

12. The cryoneedle of claim 1, wherein the outlet/inlet port is couplable to the connector by mechanical pressure, adjusted by tolerance values there between.

13. The cryoneedle of claim 1, wherein the connector further comprising one or more thermally insulating gripping elements configured to facilitate gripping of the cryoneedle by a user.

14. A method for performing a cryosurgery, the method comprising:
providing an assemblable cryoneedle according to claim 1;
contacting the assemblable cryoneedle with the treatment area of the subject or inserting the assemblable cryoneedle to the treatment area of the subject;
supplying a cryogen from a cryogenic source through the assemblable cryoneedle, such that the cryogen flows from the proximal portion to the distal portion of the inner elongated needle, back from the sealed distal portion of the outer elongated needle, and out through the outlet port of the connector; and
retracting the assemblable cryoneedle.

15. A method for performing a cryosurgery, the method comprising:
providing an assemblable cryoneedle according to claim 1;
contacting the assemblable cryoneedle with the treatment area of the subject or inserting the cryoneedle to the treatment area of the subject;
supplying a cryogen from a cryogenic source through the cryoneedle, such that the cryogen flows from the inlet port of the connector through the proximal portion of the outer elongated needle towards the distal portion of the outer elongated needle, back from the sealed distal portion of the outer elongated needle, and out through the inner elongated needle of the connector; and
retracting the assemblable cryoneedle.

16. The method of claim 14, wherein the step of retracting the assemblable cryoneedle is performed by rotating the cryoneedle clockwise to break the screwing connection (between the outer elongated needle and the connector) and render the cryoneedle a single use cryoneedle.

17. The method of claim 14, further comprising providing a non-elastic fastener configured to maintain continuous supply of the cryogen without a need of a user holding a trigger of the cryogen source in an open position.

18. The method of claim 14, further comprising inserting a trocar, preferably with a sharp sealed distal tip, into the treatment area prior to inserting the cryoneedle into the treatment area, to facilitate the cryoneedle insertion.

19. The method of claim 14, wherein the cryosurgery is performed for treating hypertrophic scars and/or keloids.

20. A kit for cryo-treatment of a subject in a need thereof, the kit comprising:
one or more assemblable cryoneedles according to claim 1; and
at least one of:
a non-elastic fastener configured to maintain continuous supply of the cryogen without a need of a user holding a trigger of the cryogen source in an open position;
a splitter for facilitating simultaneous connecting of a plurality of cryoneedles to a single cryogen source; and
a trocar, preferably with a sharp sealed distal tip, configured for introducing into the treatment area prior to inserting the cryoneedle therein, to facilitate the cryoneedle insertion.
